(19) United States
(12) Patent Application Publication
(10) Pub. No.: US 2012/0065272 A1
(43) Pub. Date: Mar. 15, 2012

(54) NOVEL USE OF PANDURATIN DERIVATIVES OR EXTRACT OF KAEMPFERIA PANDURATA COMPRISING THE SAME

(75) Inventors: Jae Kwan Hwang, Kyeonggi-Do (KR); Jae-Seok Shim, Kyeonggi-Do (KR); Song Hui Gwon, Syeonggi-Do (KR); Yi Young Kwon, Gyeonggi-Do (KR); Hyun In Oh, Gyeonggi-Do (KR); Young Sun Han, Seoul (KR); Eun Jung Choi, Seoul (KR); Do Un Kim, Gyeonggi-Do (KR)

(73) Assignee: NEWTREE CO., LTD., Sungnam, Gyeonggi-Do (KR)

(21) Appl. No.: 12/738,591

(22) PCT Filed: Oct. 17, 2008
(86) PCT No.: PCT/KR08/06137

§ 371 Date: Nov. 30, 2011

(30) Foreign Application Priority Data
Oct. 17, 2007 (KR) 10-2007-0104788

(51) Int. Cl. A61K 8/35; C07C 49/573; A61Q 19/08; C07C 49/577

(52) U.S. Cl. 514/683; 568/329

(57) ABSTRACT

The present invention relates to a new use of panduratin derivatives or a Kaempferia pandurata extract comprising the same. More particularly, the present invention relates to a composition for improving wrinkles and/or preventing aging, which comprises a panduratin derivative or a Kaempferia pandurata extract comprising the same. The said the panduratin derivative or the Kaempferia pandurata extract comprising the same induces cell proliferation, inhibits degradation of collagen, and promotes synthesis of collagen, therefore, it shows excellent activity in prevention of aging, particularly preventing, improving or treating wrinkle and it can be used as an effective ingredient in a cosmetic, food or pharmaceutical composition.

### TECHNICAL FIELD

This application claims priority based on Korean Patent Application No. 2007-0104788, filed on Oct. 17, 2007, the entire contents of which are incorporated herein by reference.

The present invention relates to a novel use of panduratin derivatives or a Kaempferia pandurata extract comprising the same. More particularly, the present invention relates to a composition for improving wrinkles and/or preventing aging, which comprises a panduratin derivative or a Kaempferia pandurata extract comprising the same.

### BACKGROUND ART

Aging is largely classified into natural aging, or intrinsic aging, and extrinsic aging. Natural aging is caused by hereditary factors and is hard to control, whereas extrinsic aging is caused by environmental factors and can be controlled relatively easily. Accordingly, researches have continued to prevent extrinsic aging. Especially, researches on the prevention of wrinkles caused by extrinsic photoaging due to long-term exposure to UV are drawing attentions [Gilchre st B. A., J. Am. Acad. Dermatol., 1989:21:610-613]. The photoaging, or extrinsic skin aging, is clinically characterized by rough and inelastic skin, irregular pigmentation and deep wrinkles.

External factors that affect the aging include wind, temperature, humidity, cigarette smoke, pollution, UV, and the like. Especially, the aging caused by UV is called photoaging. Particularly, the photoaging is deeply involved in wrinkling on face and head, which are cosmetically important areas. Therefore, basic researches on photoaging and wrinkling on human skin or in animal model are actively carried out for the development of anti-aging or anti-wrinkling cosmetics. With regard to photoaging and wrinkling, changes in basic physiological metabolism such as synthesis and degradation of collagen, the main component of skin, are reported [Lavker R. M., Blackwell Science Inc., 1995:123-135].

The photoaging mechanism will be described briefly. When the skin is exposed to a large amount of UV, a lot of reactive oxygen species are generated in the skin, disrupting the enzymatic and non-enzymatic antioxidative defense system. As a result, the content of collagen, the main protein of the skin tissue, decreases remarkably. Collagenase (matrix metalloproteinase-1; MMP-1) plays an important role in the decrease of collagen. This enzyme is involved in the degradation of the extracellular matrix and basement membrane. According to researches, exposure to UV leads to increased MMP-1 activity in the skin, thereby markedly disrupting collagen and forming wrinkles [Sim G. S., Kim J. H., et al., Kor. J. Biotechnol. Bioeng., 2005:20(1):40-45].

Some of active ingredients for improving wrinkles and preventing aging, which have been developed to date, have problems in that they cannot be used as cosmetic materials, are very unstable and are not easy to deliver to the skin. Accordingly, a special stabilizing system and delivery system are required, and the effect thereof on improving skin wrinkles is not visible. For this reason, interest in skin-protecting agents containing retinoid has recently been increased. Currently, retinoid is used as a means for solving photoaging phenomena, such as wrinkles resulting from sunlight, skin thickening, skin drooping and a decrease in skin elasticity. However, retinoid has a problem in that it is a very unstable compound, which is sensitive to UV light, moisture, heat and oxygen such that a chemical change therein easily occurs. In attempts to solve this problem, studies focused on developing effective components derived from natural resources have been conducted.

Kaempferia pandurata, also known as Boesenbergia pandurata, is a plant of the Zingiberaceae family. Its rhizome is widely used to treat cold, enteritis, skin disease and urethral pain. Kaempferia pandurata contains pinocembrin chalcone, cardamonin, pinocembrin, pinostribin, 4-hydroxypanduratin A, panduratin A, and the like. These components are reported to have anti-cancer effect [Trakoontivakorn, G., et al., J. Agri. Food Chem., 49, 3046-3050, 2001], and flavonoid-based dihydrochalcone compounds are reported to have insecticidal effect [Pandji, C., et al., Phytochemistry, 34, 415-419, 1993]. Korean Patent No. 492034 discloses an antimicrobial and an oral composition for the prevention and treatment of caries and periodontitis comprising panduratin derivatives such as isopanduratin A. However, wrinkle improvement effect or anti-aging effect of panduratin derivatives or a Kaempferia pandurata extract comprising the same have never been reported yet.

### DISCLOSURE

### Technical Problem

The inventors of the present invention have researched on natural substances having wrinkle improvement or anti-aging activity. They found out that panduratin derivatives or a Kaempferia pandurata extract comprising the same have such an activity and accomplished the present invention.

Accordingly, in an aspect, the present invention provides a cosmetic composition for wrinkle improvement and/or anti-aging comprising a panduratin derivative selected from the group consisting of the compounds represented by the following Chemical Formulas 1 to 3 or a Kaempferia pandurata extract comprising the same:

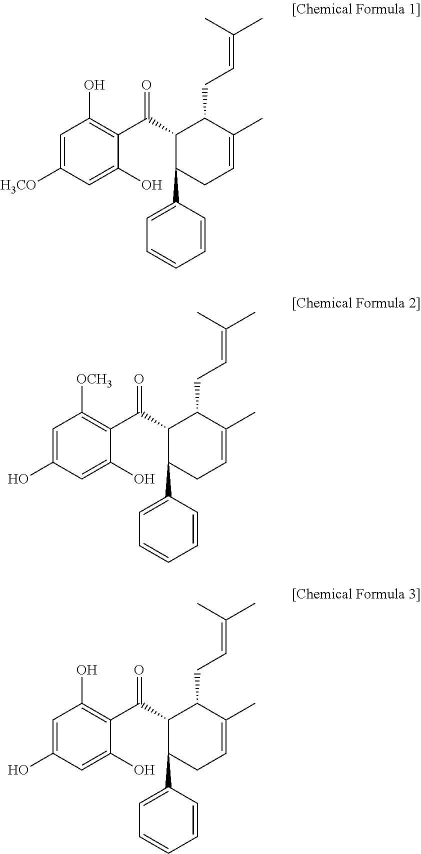

In another aspect, the present invention provides a food composition for wrinkle improvement and/or anti-aging comprising a panduratin derivative selected from the group consisting of the compounds represented by the Chemical Formulas 1 to 3 or a Kaempferia pandurata extract comprising the same.

In another aspect, the present invention provides a pharmaceutical composition for preventing and treating wrinkle and/or preventing aging comprising a panduratin derivative selected from the group consisting of the compounds represented by the Chemical Formulas 1 to 3 or a Kaempferia pandurata extract comprising the same.

In another aspect, the present invention provides a use of a panduratin derivative selected from the group consisting of the compounds represented by the Chemical Formulas 1 to 3 or a Kaempferia pandurata extract comprising the same for preparing a cosmetic composition.

In another aspect, the present invention provides a use of a panduratin derivative selected from the group consisting of the compounds represented by the Chemical Formulas 1 to 3 or a Kaempferia pandurata extract comprising the same for preparing a food composition.

In another aspect, the present invention provides a use of a panduratin derivative selected from the group consisting of the compounds represented by the Chemical Formulas 1 to 3 or a Kaempferia pandurata extract comprising the same for preparing a pharmaceutical composition.

In another aspect, the present invention provides a method for preventing, improving or treating wrinkle of a panduratin derivative selected from the group consisting of the compounds represented by the Chemical Formulas 1 to 3 or a Kaempferia pandurata extract comprising the same.

In another aspect, the present invention provides a method for preventing aging of a panduratin derivative selected from the group consisting of the compounds represented by the Chemical Formulas 1 to 3 or a Kaempferia pandurata extract comprising the same.

In another aspect, the present invention provides a method for promoting collagen synthesis of a panduratin derivative selected from the group consisting of the compounds represented by the Chemical Formulas 1 to 3 or a Kaempferia pandurata extract comprising the same.

In another aspect, the present invention provides a method for inhibiting collagen degradation of a panduratin derivative selected from the group consisting of the compounds represented by the Chemical Formulas 1 to 3 or a Kaempferia pandurata extract comprising the same.

### Technical Solution

To achieve the above objects, the present invention provides a cosmetic composition for wrinkle improvement and/or anti-aging comprising a panduratin derivative selected from the group consisting of the compounds represented by the following Chemical Formulas 1 to 3 or a Kaempferia pandurata extract comprising the same:

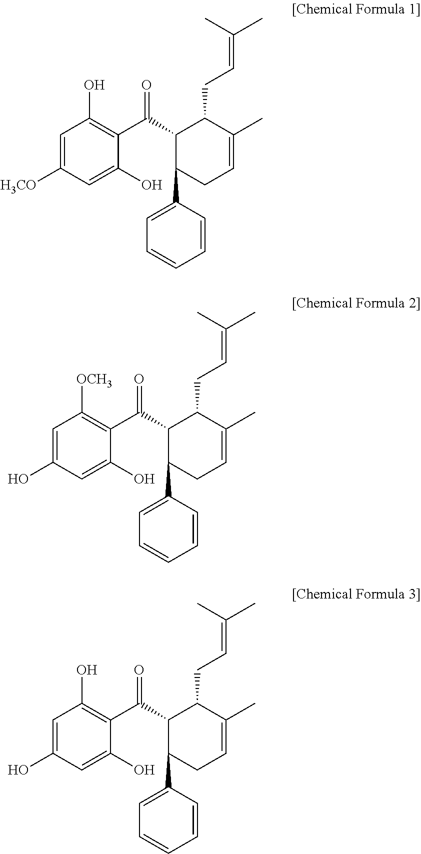

The present invention provides a food composition for wrinkle improvement and/or anti-aging comprising a panduratin derivative selected from the group consisting of the compounds represented by the Chemical Formulas 1 to 3 or a Kaempferia pandurata extract comprising the same.

The present invention provides a pharmaceutical composition for preventing and treating wrinkle and/or preventing aging comprising a panduratin derivative selected from the group consisting of the compounds represented by the Chemical Formulas 1 to 3 or a Kaempferia pandurata extract comprising the same.

The present invention provides a use of a panduratin derivative selected from the group consisting of the compounds represented by the Chemical Formulas 1 to 3 or a Kaempferia pandurata extract comprising the same for preparing a cosmetic composition.

The present invention provides a use of a panduratin derivative selected from the group consisting of the compounds represented by the Chemical Formulas 1 to 3 or a Kaempferia pandurata extract comprising the same for preparing a food composition.

The present invention provides a use of a panduratin derivative selected from the group consisting of the compounds represented by the Chemical Formulas 1 to 3 or a Kaempferia pandurata extract comprising the same for preparing a pharmaceutical composition.

The present invention provides a method for preventing, improving or treating wrinkle of a panduratin derivative selected from the group consisting of the compounds represented by the Chemical Formulas 1 to 3 or a Kaempferia pandurata extract comprising the same.

The present invention provides a method for preventing aging of a panduratin derivative selected from the group consisting of the compounds represented by the Chemical Formulas 1 to 3 or a Kaempferia pandurata extract comprising the same.

The present invention provides a method for promoting collagen synthesis of a panduratin derivative selected from the group consisting of the compounds represented by the Chemical Formulas 1 to 3 or a Kaempferia pandurata extract comprising the same.

The present invention provides a method for inhibiting collagen degradation of a panduratin derivative selected from the group consisting of the compounds represented by the Chemical Formulas 1 to 3 or a Kaempferia pandurata extract comprising the same.

Hereafter, the present invention will be described in detail.

The “Kaempferia pandurata extract” disclosed in the present invention refers to an extract obtained from Kaempferia pandurata, also known as Boesenbergia pandurata, comprising the aforesaid panduratin derivative. The method for preparing the Kaempferia pandurata extract is not specially limited as long as the panduratin derivative is included in the extract. Preferably, it may be prepared by extracting the whole plant or part (stem, rhizome or leaf) of Kaempferia pandurata (Roxb.) with at least one solvent selected from the group consisting of water, C₁-C₆ organic solvent and subcritical or supercritical fluid. If necessary, a process of filtration or condensation commonly used in the related art may be further added.

The C₁-C₆ organic solvent may be selected from C₁-C₆ alcohol, acetone, ether, benzene, chloroform, ethyl acetate, methylene chloride, hexane, cyclohexane and petroleum ether, but not limited thereto.

As used herein, the “supercritical fluid” refers to any substance at a temperature and pressure above its thermodynamic critical point. The “subcritical fluid” includes subcritical liquid and gas. Especially, the subcritical liquid refers to a fluid at temperatures below the supercritical temperature and the saturation temperature. And, the subcritical gas refers to a fluid at temperatures above the saturation temperature and pressures below the supercritical pressure. The supercritical fluid and subcritical fluid are used in various fields, including pharmaceutical industry, food industry, cosmetics/perfume industry, chemical industry and energy industry. The supercritical fluid and subcritical fluid that may be used in the present invention are not specially limited. For example, carbon dioxide, nitrogen, nitrous oxide, methane, ethylene, propane, propylene, petroleum ether, ethyl ether, cyclohexane, etc. may be used. Especially, carbon dioxide is preferred because it is easily available, relatively inexpensive, inexplosive, and sufficiently safe for processing. Carbon dioxide has a critical temperature of 31.1° C. and a critical pressure of 73.8 atm.

As an embodiment of the present invention, dry Kaempferia pandurata was ground, extracted using ethanol, hexane or chloroform solvent, filtered and concentrated to prepare an ethanol, hexane or chloroform extract of Kaempferia pandurata. Further, Kaempferia pandurata was added to a supercritical fluid extractor using carbon dioxide (CO₂) as supercritical fluid to prepare a supercritical extract of Kaempferia pandurata (see Example 1).

As used herein, the “panduratin derivative” refers to a compound selected from the group consisting of the compounds represented by the following Chemical Formulas 1 to 3. Specifically, the compounds represented by Chemical Formulas 1, 2 and 3 are panduratin A, isopanduratin A and 4-hydroxypanduratin A, respectively.

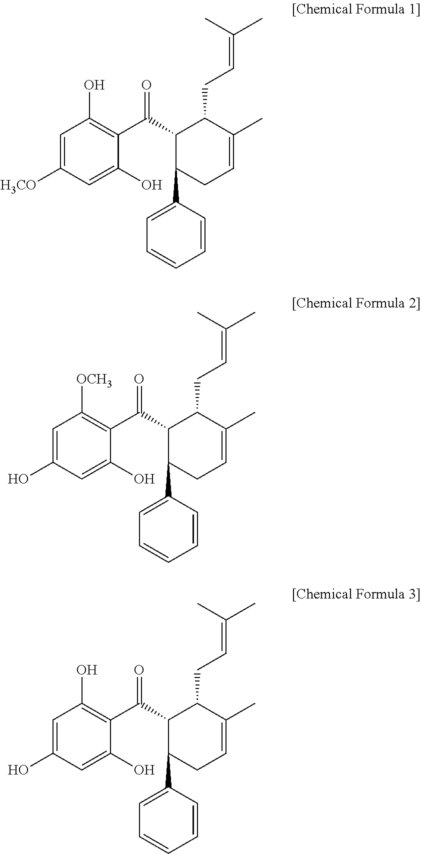

The panduratin derivative is commercially available or may be prepared according to a known synthesis method. It may be prepared by separating and purifying a Kaempferia pandurata extract or oil obtained by pressing Kaempferia pandurata. For the separation and purification of the panduratin derivative from the Kaempferia pandurata extract, column chromatography or high-performance liquid chromatography (HPLC) using silica gel, activated alumina or various other synthetic resins may be used alone or in combination, although not limited thereto.

As an embodiment of the present invention, dry Kaempferia pandurata was ground and mixed with ethanol. After solvent extraction, the solvent was removed and the resulting extract was concentrated. The concentrated crude extract was mixed with ethyl acetate. After extracting the ethyl acetate soluble component followed by the removal of ethyl acetate, the ethyl acetate soluble component was concentrated and separated depending on polarities. Specifically, at first, development was carried out using a mixture solvent of hexane and ethyl acetate to remove impurities. Then, separation was carried out using a mixture solvent of hexane, chloroform and ethyl acetate. Finally, panduratin A, isopanduratin A or 4-hydroxypanduratin A was obtained (see FIG. 1 and Examples 2 through 4).

The panduratin derivative selected from the group consisting of the compounds represented by Chemical Formulas 1 to 3 or the Kaempferia pandurata extract comprising the same has superior anti-aging activity and is excellent in improving, preventing or treating wrinkle.

Specifically, the panduratin derivative selected from the group consisting of the compounds represented by the Chemical Formulas 1 to 3 or the Kaempferia pandurata extract comprising the same has superior cell proliferation activity. When MTT assay was carried out using fibroblasts, the addition of the panduratin derivative selected from the group consisting of the compounds represented by the Chemical Formulas 1 to 3 or the Kaempferia pandurata extract comprising the same induced cell proliferation markedly (see Test Example 1).

Further, the panduratin derivative selected from the group consisting of the compounds represented by the Chemical Formulas 1 to 3 or the Kaempferia pandurata extract comprising the same has the activity of inhibiting collagen degradation by suppressing the expression of MMP-1, and promoting collagen synthesis by inducing the synthesis of procollagen. When treated with the panduratin derivative selected from the group consisting of the compounds represented by the Chemical Formulas 1 to 3 or the Kaempferia pandurata extract comprising the same, the expression of MMP-1 was suppressed whereas the procollagen synthesis increased (see Test Example 2).

The inventors have revealed the mechanism by which the panduratin derivative selected from the group consisting of the compounds represented by the Chemical Formulas 1 to 3 or the Kaempferia pandurata extract comprising the same inhibits collagen degradation and promotes collagen synthesis.

Specifically, collagen degradation occurs by the following mechanism. When extracellular-regulated protein kinase (ERK), Jun-N-terminal kinase (JNK) and p38 kinase, which belong to mitogen-activated protein kinases (MAPKs), are activated by phosphorylation, activation of activator protein-1 (AP-1) is induced [Xu Y, Fisher G J., J. Dermatol. Sci. Suppl. 2005; 1: S1 S8], resulting in binding with DNA. Through this, MMPs (matrix metalloproteinase) are excreted and collagen is degraded. Here, c-Jun and c-Fos are known to play a role in the binding of AP-1 with DNA [Waskiewicz A J, Cooper J A., Curr. Opin. Cell Biol., 1995; 7: 798 805].

When panduratin A was added, the activity of ERK, JNK and p38 kinase, and the binding of AP-1 with DNA were suppressed. Further, the activity of c-Jun and c-Fos was suppressed. As a result, the secretion of MMPs was suppressed. Accordingly, panduratin A was confirmed to have the activity of inhibiting collagen degradation and promoting collagen synthesis (see Test Example 3).

Further, when applied on the skin or administered orally to mice in which wrinkling was induced by exposure to UV, the panduratin derivative selected from the group consisting of the compounds represented by the Chemical Formulas 1 to 3 or the Kaempferia pandurata extract comprising the same exhibited a remarkable wrinkle improvement effect (see Test Example 4).

Accordingly, the panduratin derivative selected from the group consisting of the compounds represented by the Chemical Formulas 1 to 3 or the Kaempferia pandurata extract comprising the same is excellent in preventing aging, and particularly, excellent in preventing, improving or treating wrinkle, thereby being used as an effective component of cosmetic, food or pharmaceutical composition.

A composition for cosmetics of the present invention contains the panduratin derivative selected from the group consisting of the compounds represented by the Chemical Formulas 1 to 3 or the Kaempferia pandurata extract comprising the same as an effective component, and may be prepared in the form of basic cosmetics (lotions, cream, essence, cleansers such as cleansing foam and cleansing water, pack, body oil), coloring cosmetics (foundation, lip-stick, mascara, make-up base), hair care composition (shampoo, rinse, hair conditioner, hair gel) and soap with dermatologically acceptable excipients.

The said excipients may comprise, but not limited thereto, skin softener, skin infiltration enhancer, colorant, odorant, emulsifier, thickener, or solvent. In addition, it is possible to add fragrance, a pigment, bactericidal agent, an antioxidant, a preservative, moisturizer and the like, and to add thickening agents, inorganic salts or synthetic polymers for improving physical properties. For example, in case of manufacturing a cleanser and soap comprising composition of the present invention, they may be prepared easily by adding the panduratin derivative or the Kaempferia pandurata extract comprising the same to conventional cleanser or soap base. In case of manufacturing a cream, it may be prepared by adding the panduratin derivative or the Kaempferia pandurata extract comprising the same to conventional oil-in-water cream base. In addition, it is possible to add a fragrance, a chelating agent, a pigment, an antioxidant, a preservative, and the like, and to add proteins, minerals or synthetic polymers for improving physical properties.

The panduratin derivative or the Kaempferia pandurata extract comprising the same of the present invention may be preferably comprised by the form of composition for cosmetics in the range of 0.001-10 wt %, and more preferably 0.01-5 wt %, based on the total weight of a formulation. If the composition is added in an amount of less than 0.001 wt %, it will provide low effect in preventing aging or improving wrinkle, and if it is added in an amount of more than 10 wt %, it will have a difficulty in safety or formulation.

Meanwhile, The composition for food of the present invention may comprise all kinds of forms including functional food, nutritional supplement, health food, and food additives.

The said composition for food may be prepared into various kinds of forms by the methods known in the art.

For example, as a health food, the panduratin derivative or the Kaempferia pandurata extract comprising the same of the present invention may be prepared into tea, juice, and drink for drinking or may be prepared into granules, capsules, or powder for uptake. Also, conventional active ingredient which is well known as having activity in prevention of aging or prevention, improvement or treatment of wrinkle may be mixed with the panduratin derivative or the Kaempferia pandurata extract comprising the same of the present invention so as to prepare a composition. Also, for preparing functional foods, the panduratin derivative or the Kaempferia pandurata extract comprising the same of the present invention may be added to beverages (including alcoholic beverages), fruits, and their processed foods (e.g. canned fruit, bottled fruit, jam, marmalade etc.), fishes, meats, and their processed foods (e.g. ham, sausage, corn beef etc.), breads and noodles (e.g. Japanese noodle, buckwheat noodle, Chinese noodle, spaghetti, macaroni etc.), fruit juice, drinks, cookies, toffee, dairy products (e.g. butter, cheese etc.), vegetable oil, margarine, vegetable protein, retort food, frozen food, various seasonings (e.g. soybean paste, soybean sauce, sauce etc.). In addition, the panduratin derivative or the Kaempferia pandurata extract comprising the same may be prepared in a form of powder or extract for food additives.

The panduratin derivative or the Kaempferia pandurata extract comprising the same of the present invention may be properly comprised by the form of composition for food preferably in the range of 0.01 to 50% based on the total weight of a food. More preferably, a food composition of the present invention may be prepared particularly mixing conventional active ingredient which is well known as having activity in prevention of aging or prevention, improvement or treatment of wrinkle with the panduratin derivative or Kaempferia pandurata extract comprising the same of the present invention.

Meanwhile, a pharmaceutical composition of the present invention may comprise the panduratin derivative or the Kaempferia pandurata extract comprising the same alone or together with one or more carrier, excipient, or diluent additionally.

A pharmaceutically acceptable carrier, for example, carriers for the parenteral or oral preparations may be included. The carriers for the oral preparations may comprise lactose, starch, cellulose derivatives, magnesium stearate, stearic acid. In addition, the carriers for the parenteral preparations may comprise water, oil, saline, aqueous glucose and glycol, and stabilizers and preservatives. The examples of the stabilizers may be antioxidant such as sodium hydrogen sulfite, sodium sulfite, and ascorbic acid. The examples of the preservatives may be benzalkonium chloride, methyl- or prophyl-paraben, and chlorobutanol. The list of pharmaceutically acceptable carriers are disclosed in Remington's Pharmaceutical Sciences, 19th ed., Mack Publishing Company, Easton, Pa., 1995.

The inventive pharmaceutical composition may be administered to mammalians including human beings by various routes. For example, it may be administered by oral or parenteral preparation. A parenteral preparation may be, but not limited thereto, intravenous, intramuscular, intraarterial, intramarrow, subdural, intracardiac, intracutaneous, subcutaneous, intraperitoneal, intranasal, gastrointestinal tracts, parenteral, sublingual or rectum. A pharmaceutical composition of the present invention may be prepared in the form of oral preparation or parenteral preparation according to the described above. In case of the formulation for oral administration, the composition of the present invention may be formulated into powders, granules, tablets, pills, and sugar-coated tablets, capsules, liquids, gels, syrups, slurries, and emulsions by the method well known in the art. For example, preparations for oral administration may be harvested in the form of tablets or sugar-coated tablets by mixing an effective component with a solid excipient, grinding, and adding appropriate supplemental agents, then manufacturing a form of granular mixture. For examples of appropriate excipient, it may comprise sugars comprising lactose, dextrose, sucrose, sorbitol, mannitol, xylitol, erythritol and maltitol, starches comprising corn starch, wheat starch, rice starch and potato starch, celluloses comprising cellulose, methyl cellulose, sodium carboxymethylcellulose and hydroxypropylmethylcellulose, and fillers comprising gelatin and polyvinylpyrrolidone. And, if desired, it may comprise cross-linked polyvinylpyrrolidone, agar, alginic acid or sodium alginate as a solutionizer. Further, the inventive pharmaceutical composition may comprise anti-coagulant, lubricant, wetting agents, flavors, emulsifying agents and antiseptics additionally. In case of pharmaceutical formulations for parenteral administration, it may be prepared in the forms of injectable preparations, creams, lotions, ointments, oils, humectant, gels, aerosol, and nasal inhalations by the method well known in the art. The formulation of the above-mentioned is well described in Remington's Pharmaceutical Science, 15th Edition, 1975. Mack Publishing Company, Easton, Pa. 18042, Chapter 87: Blaug, Seymour which is well known prescription book.

Total effective amount of pharmaceutical composition of the present invention may be administered to a patient with a single dose, or may be administered with multiple doses by fractionated treatment protocol. The pharmaceutical composition of the present invention may contain variable amount of effective ingredient according to the disease severity. In case of parenteral administration, the effective amount of the panduratin derivative or the Kaempferia pandurata extract comprising the same is preferably about 0.01 to 50 mg/kg body weight/day, more preferably 0.1 to 30 mg/kg body weight/day, and, in case of oral administration, is preferably about 0.01 to 100 mg/kg body weight/day, more preferably 0.1 to 50 mg/kg body weight/day with a single dose or multiple doses. However, the dose of panduratin derivative or Kaempferia pandurata extract comprising the same may be suitably determined by considering various factors, such as age, body weight, health condition, sex, disease severity, diet and excretion of a subject in need of treatment, as well as administration time and administration route. When those are considered, skilled person in the art may determine appropriate dose of the panduratin derivative or the Kaempferia pandurata extract comprising the same for a certain use for the prevention of aging or prevention, improvement or treatment of wrinkle. The inventive pharmaceutical compositions may not limit formulations, administration routes, and administration methods as long as they show the effect of the present invention

Further, because the panduratin derivative selected from the group consisting of the compounds represented by the Chemical Formulas 1 to 3 or the Kaempferia pandurata extract comprising the same has superior anti-aging activity and is especially excellent in improving, preventing or treating wrinkle, it can be used for preparing a cosmetic composition, food composition or pharmaceutical composition for anti-aging or improving, preventing or treating wrinkle.

The method for preparing the cosmetic composition, food composition or pharmaceutical composition using the panduratin derivative or the Kaempferia pandurata extract is the same as described above. And, the content of the panduratin derivative or the Kaempferia pandurata extract in the compositions is the same as described above.

Further, the panduratin derivative selected from the group consisting of the compounds represented by the Chemical Formulas 1 to 3 or the Kaempferia pandurata extract comprising the same may be used in a method for preventing, improving or treating wrinkle, or for preventing aging.

For preventing, improving or treating wrinkle, or for preventing aging, the panduratin derivative or the Kaempferia pandurata extract may be administered to a subject in need thereof with an effective amount.

As used herein, the “subject in need” refers to a mammal, preferably a human, in need of preventing, improving or treating wrinkle, or preventing aging. And, the “effective amount” refers to an amount which exhibits the effect of preventing, improving or treating wrinkle, and preventing aging by inhibiting collagen degradation and promoting collagen synthesis in the subject. The administration method and administration dose for administering with the effective amount are the same as described in detail above.

Further, the panduratin derivative selected from the group consisting of the compounds represented by the Chemical Formulas 1 to 3 or the Kaempferia pandurata extract comprising the same may be used in a method for promoting collagen synthesis and inhibiting collagen degradation.

The activity of collagen synthesis promotion and collagen degradation inhibition of the panduratin derivative or the Kaempferia pandurata extract and the mechanism thereof are the same as described above.

### Advantageous Effects

As can be seen from the foregoing, the panduratin derivative selected from the group consisting of the compounds represented by the Chemical Formulas 1 to 3 or the Kaempferia pandurata extract comprising the same induces cell proliferation, inhibits degradation of collagen, and promotes synthesis of collagen, therefore, it shows excellent activity in preventing aging, particularly preventing, improving or treating wrinkle and it can be used as an effective ingredient in a cosmetic, food or pharmaceutical composition.

### DESCRIPTION OF DRAWINGS

FIG. 1 illustrates a process of isolating the substances having wrinkle improvement activity from Kaempferia pandurata.

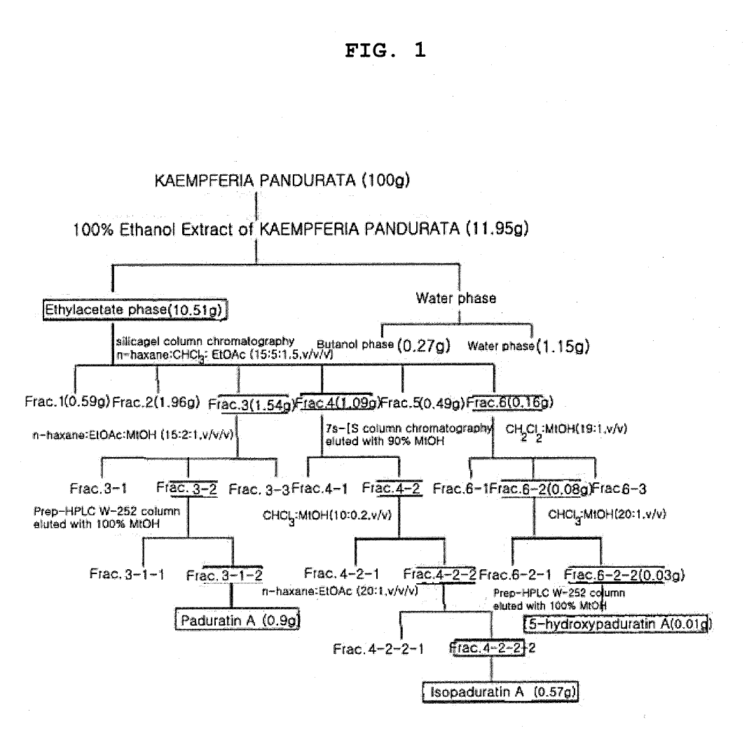

FIG. 2 shows MMP-1 inhibition activity and procollagen synthesis promotion activity of the ethanol extract of Kaempferia pandurata (A: MMP-1 expression inhibition activity, B: MMP-1 mRNA expression inhibition activity, C: procollagen biosynthesis promotion activity, D: procollagen mRNA expression promotion activity).

FIG. 3 shows MMP-1 inhibition activity and procollagen synthesis promotion activity of panduratin A (A: MMP-1 expression inhibition activity, B: MMP-1 mRNA expression inhibition activity, C: procollagen biosynthesis promotion activity, D: procollagen mRNA expression promotion activity).

FIG. 4 shows MMP-1 inhibition activity and procollagen synthesis promotion activity of isopanduratin A (A: MMP-1 expression inhibition activity, B: MMP-1 mRNA expression inhibition activity, C: procollagen biosynthesis promotion activity, D: procollagen mRNA expression promotion activity).

FIG. 5 shows MMP-1 inhibition activity and procollagen synthesis promotion activity of 4-hydroxypanduratin (A: MMP-1 expression inhibition activity, B: MMP-1 mRNA expression inhibition activity, C: procollagen biosynthesis promotion activity, D: procollagen mRNA expression promotion activity).

FIG. 6 shows the effect of the Kaempferia pandurata extract or the panduratin derivatives on activation of MAPKs.

FIG. 7 shows the effect of the Kaempferia pandurata extract or the panduratin derivatives on the DNA binding activity of AP-1.

FIG. 8 shows the effect of the Kaempferia pandurata extract or the panduratin derivatives on the c-Jun and c-Fos activity.

FIG. 9 shows skin replicas of mice after application of the Kaempferia pandurata extract or the panduratin derivatives.

FIG. 10 shows Rt, Rm, Rz and Ra measurement result after application of the Kaempferia pandurata extract or the panduratin derivatives on the skin of mice (Rt: the distance from the highest and lowest portions on the skin surface, Rm: the maximum Rt value of 5 measurements, Rz: the mean Rt value of 5 measurements, Ra: the arithmetic mean surface roughness).

FIG. 11 shows skin replicas of mice after oral administration of the Kaempferia pandurata extract or the panduratin derivatives.

### MODE FOR INVENTION

Hereinafter, the constitution and effect of the present invention will be described in detail through examples and test examples. However, the following examples and test examples are given only for the purpose of illustrating the present invention, and the scope of the present invention is not limited by them.

### Example 1

### Preparation of Kaempferia pandurata Extract Comprising Panduratin

1-1. Preparation of Ethanol Extract of Kaempferia pandurata

Dry Kaempferia pandurata rhizome was ground using a mixer. 100 g of the ground Kaempferia pandurata sample was added to 1 L of ethanol and extracted at room temperature for 48 hours. The extracted sample was filtered through Whatman No. 2 filter paper. The solvent component was removed from the filtered extract solution by concentrating using a vacuum rotary evaporator. An ethanol extract of Kaempferia pandurata was obtained.

1-2. Preparation of Hexane Extract of Kaempferia pandurata

Dry Kaempferia pandurata rhizome was ground using a mixer. 100 g of the ground Kaempferia pandurata sample was added to 1 L of hexane and extracted at room temperature for 48 hours. The extracted sample was filtered through Whatman No. 2 filter paper. The solvent component was removed from the filtered extract solution by concentrating using a vacuum rotary evaporator. A hexane extract of Kaempferia pandurata was obtained.

1-3. Preparation of Chloroform Extract of Kaempferia pandurata

Dry Kaempferia pandurata rhizome was ground using a mixer. 100 g of the ground Kaempferia pandurata sample was added to 1 L of chloroform and extracted at room temperature for 48 hours. The extracted sample was filtered through Whatman No. 2 filter paper. The solvent component was removed from the filtered extract solution by concentrating using a vacuum rotary evaporator. A chloroform extract of Kaempferia pandurata was obtained.

1-4. Preparation of Supercritical Extract of Kaempferia pandurata

A supercritical extract was obtained from Kaempferia pandurata rhizome using a supercritical fluid extractor and using carbon dioxide (CO₂) as supercritical fluid. After extracting at 50° C. and 200 bar, the solvent component was removed from the extract solution. A supercritical extract was obtained.

### Example 2

### Isolation of Panduratin A

The concentrated ethanol extract of Kaempferia pandurata obtained in Example 1-1 was mixed with ethyl acetate. The ethyl acetate soluble component was extracted and ethyl acetate was removed under reduced pressure to concentrate the ethyl acetate soluble component. Using a column in which silica gel was packed with 6×15 cm, and using a solvent system consisting of n-hexane, chloroform and ethyl acetate (15:5:1.5, v/v/v), a total of 6 fractions were concentrated and dried. Of the 6 fractions, the 3rd fraction (fraction 3) was subjected to thin layer chromatography (TLC, silica gel 60F254, Merck) using a developing solvent consisting of n-hexane, ethyl acetate and methanol (18:2:1, v/v/v). A total of 3 fractions were concentrated and dried. Of the 3 fractions, the 2nd fraction (fraction 3-2) was subjected to recycling HPLC (column: W-252, 20.0 mm ID×500 mm L). A total of 2 fractions were concentrated and dried. Finally, of the 2 fractions, the 2nd fraction (fraction 3-2-2) was concentrated and dried. Panduratin A of the following Chemical Formula 1 was isolated as a pure substance having wrinkle improvement activity.

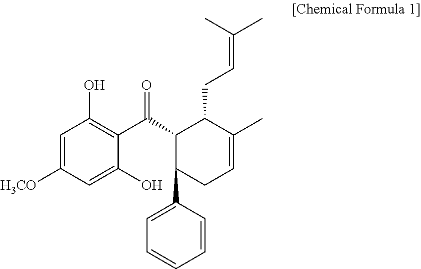

### Example 3

### Isolation of Isopanduratin A

The concentrated ethanol extract of Kaempferia pandurata obtained in Example 1-1 was mixed with ethyl acetate. The ethyl acetate soluble component was extracted and ethyl acetate was removed under reduced pressure to concentrate the ethyl acetate soluble component. Using a column in which silica gel was packed with 6×15 cm, and using a solvent system consisting of n-hexane, chloroform and ethyl acetate (15:5:1.5, v/v/v), a total of 6 fractions were concentrated and dried. Of the 6 fractions, the 4th fraction (fraction 4) was eluted using a reverse phase-18 (Rp-18, LiChropep, 25-40 m) packing material and using a solvent system consisting of methanol and water (9:1, v/v). A total of fractions were obtained. Of the 2 fractions, the 2nd fraction (fraction 4-2) was concentrated, dried and eluted using a solvent system consisting of chloroform and methanol (10:0.2, v/v). A total of 2 fractions were concentrated and dried. Of the 2 fractions, the 2nd fraction (fraction 4-2-2) was eluted using a solvent system consisting of n-hexane and ethyl acetate (10:3, v/v). A total of 2 fractions were concentrated and dried. Finally, of the 2 fractions, the 2nd fraction (fraction 4-2-2-2) was concentrated and dried. Isopanduratin A of the following Chemical Formula 2 was isolated as a pure substance having wrinkle improvement activity.

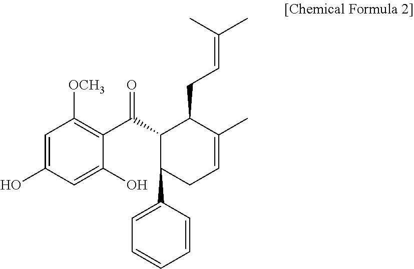

### Example 4

### Isolation of 4-Hydroxypanduratin A

The concentrated ethanol extract of Kaempferia pandurata obtained in Example 1-1 was mixed with ethyl acetate. The ethyl acetate soluble component was extracted and ethyl acetate was removed under reduced pressure to concentrate the ethyl acetate soluble component. Using a column in which silica gel was packed with 6×15 cm, and using a solvent system consisting of n-hexane, chloroform and ethyl acetate (15:5:1.5, v/v/v), a total of 6 fractions were concentrated and dried. Of the 6 fractions, the 6th fraction (fraction 6) was eluted using a solvent system consisting of methylene chloride and methanol (19:1, v/v). A total of 3 fractions were obtained. Of the 3 fractions, the 2nd fraction (fraction 6-2) was eluted using a solvent system consisting of chloroform and methanol (20:1, v/v). A total of 2 fractions were obtained. Finally, of the 2 fractions, the 2nd fraction (fraction 6-2-2) was subjected to recycling HPLC (column: W-252, 20.0 mm ID×500 mm L). 4-Hydroxypanduratin A of the following Chemical Formula 3 was isolated as a pure substance having wrinkle improvement activity.

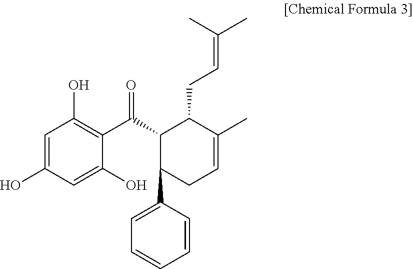

### Test Example 1

### Cell Proliferation Effect of Kaempferia pandurata Extract and Panduratin Derivatives

1-1. Cell Proliferation of Kaempferia pandurata Extract

MTT assay [3-(4,5-dimethylthiazol-2-yl)-2,5-diphenyltetrazolium bromide reduction method] was performed on fibroblasts to evaluate cell proliferation of the Kaempferia pandurata ethanol extract prepared in Example 1-1. A green tea extract, which is known to have skin wrinkle improvement effect, was selected as control substance. The result is given in the following Table 1.

TABLE 1
Cell proliferation effect of Kaempferia pandurata extract

| | Proliferation | |
|---|---|---|
| | effect (%) | Proliferation |
| Concentration | Kaempferia | effect (%) |
| (μg/mL) | pandurata extract | Green tea extract |
| 0 | 100 | 100 |
| 0.001 | 102 | 101 |
| 0.01 | 103 | 102 |
| 0.1 | 107 | 105 |
| 1 | 121 | 119 |
| 10 | 128 | 121 |
| 50 | 134 | 128 |

As seen from Table 1, the Kaempferia pandurata extract of the present invention exhibited better cell proliferation effect than the control substance.

### 1-2. Cell Proliferation of Panduratin Derivatives

MTT assay [3-(4,5-dimethylthiazol-2-yl)-2,5-diphenyltetrazolium bromide reduction method] was performed on fibroblasts to evaluate cell proliferation of the panduratin derivatives prepared in Examples 2 through 4. Epigallocatechin-3-O-gallate (EGCG), which is known to have skin wrinkle improvement effect, was selected as control substance. The result is given in the following Table 2.

TABLE 2
Cell proliferation effect of panduratin derivatives

| | | Proliferation | Proliferation | |
|---|---|---|---|---|
| | Proliferation | effect (%) | effect (%) | Proliferation |
| Concentration | effect (%) | Isopan- | 4-Hydroxy- | effect (%) |
| (μM) | Panduratin A | duratin A | panduratin A | EGCG |
| 0 | 100 | 100 | 100 | 100 |
| 0.001 | 103 | 102 | 101 | 102 |
| 0.01 | 104 | 102 | 102 | 104 |
| 0.1 | 111 | 110 | 109 | 107 |
| 1 | 120 | 120 | 119 | 119 |
| 10 | 126 | 124 | 121 | 120 |
| 50 | 131 | 130 | 128 | 126 |

As seen from Table 2, the panduratin derivatives of the present invention exhibited better cell proliferation effect than the control substance.

### Test Example 2

### MMP-1 Inhibition and Procollagen Synthesis Promotion Effect of Kaempferia pandurata Extract or Panduratin Derivatives

2-1. Kaempferia pandurata Extract

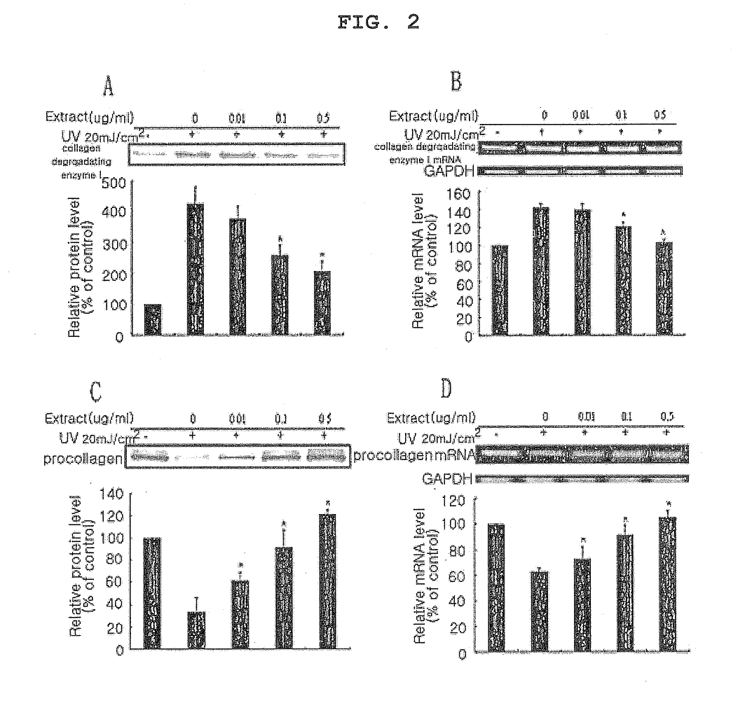

MMP-1 inhibition activity and procollagen synthesis promotion effect of the Kaempferia pandurata ethanol extract prepared in Example 1 were measured by Western blotting and reverse transcriptase-polymerase chain reaction (RT-PCR). The result is given in FIG. 2.

Specifically, proteins were extracted from cultured fibroblasts and quantitated using a protein assay reagent (Bio-Rad Laboratories Inc., Hercules, Calif., USA). For Western blotting, the proteins were heated for 3 minutes and, after cooling, transferred to a nitrocellulose membrane (Amersham International, Little Chalfont, England) through electrophoresis in 10% SDS-PAGE. The membrane was saturated with 5% skim milk in TBST (10 mM Tris, pH 7.5, 100 mM NaCl, 0.1% Tween 20). After blotting for 2 hours using primary antibodies (diluted to 1:1000), followed by washing with TBST, blotting was performed for 2 hours using secondary antibodies (diluted to 1:2000). After washing with TBST for 3 times, the blotted antibodies were analyzed using an ECL detection system (Amersham International, Little Chalfont, England).

For RT-PCR, RNAs were isolated from fibroblasts using TRIZOL (Invitrogen, USA). The isolated RNAs were quantitated using MMP-1 and procollagen primers and Taq polymerase. Specifically, RT-PCR was carried out using MMP-1 primer for 25 cycles, each cycle consisting of 30 seconds at 94° C., 1 minute at 50° C. and 1 minute at 72° C. And RT-PCR was carried out using procollagen primer for 28 cycles, each cycle consisting of 30 seconds at 94° C., 1 minute at 60° C. and 1 minute at 72° C. Then, after electrophoresis using 1% agarose gel, expression of mRNA for MMP-1 and procollagen was analyzed through ethidium bromide (EtBr) illumination.

As seen from FIG. 2, treatment with the Kaempferia pandurata ethanol extract resulted in the decrease of expression of MMP-1 proteins and mRNAs in a concentration-dependent manner, whereas expression of procollagen proteins and mRNAs increased in a concentration-dependent manner (see FIG. 2).

The same experiments were carried out for the hexane extract, chloroform extract and supercritical extract of Kaempferia pandurata prepared in Example 1. Treatment with the Kaempferia pandurata hexane extract resulted in 37% decrease of MMP-1 expression and 250% increase of procollagen synthesis as compared to the control group (not shown in the figure). And, treatment with the Kaempferia pandurata chloroform extract resulted in 40% decrease of MMP-1 expression and 290% increase of procollagen synthesis as compared to the control group (not shown in the figure). At last, treatment with the Kaempferia pandurata supercritical extract resulted in 29% decrease of MMP-1 expression and 220% increase of procollagen synthesis as compared to the control group (not shown in the figure).

Thus, it was confirmed that the Kaempferia pandurata extract can be effectively used for anti-aging or wrinkle improvement because it inhibits the expression of collagenase and promotes procollagen synthesis.

### 2-2. Panduratin Derivatives

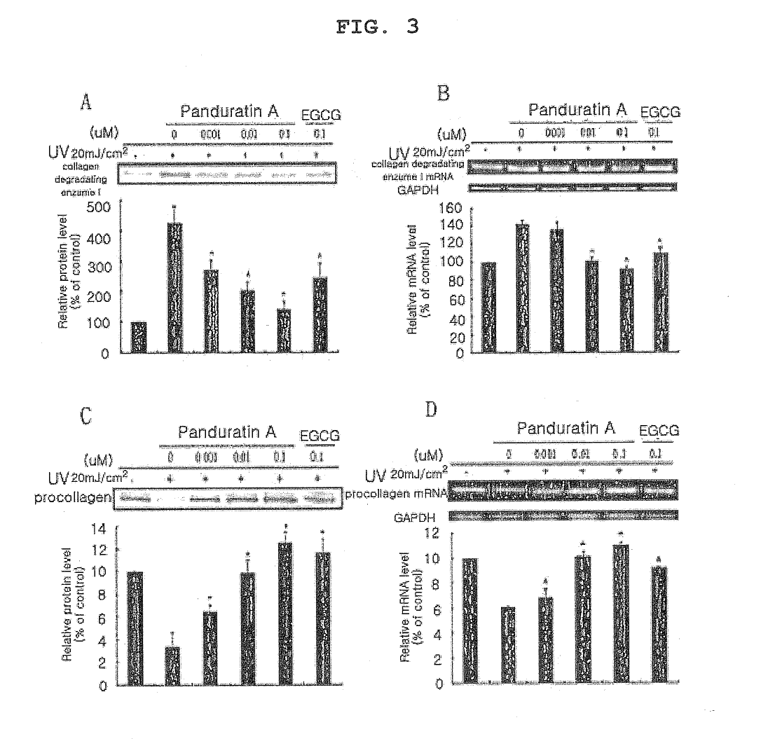

MMP-1 inhibition activity and procollagen synthesis promotion effect of the panduratin derivatives prepared in Examples 2 through 4 were measured by Western blotting and RT-PCR in the same manner as Test Example 2-1. The result is given in FIGS. 3-5.

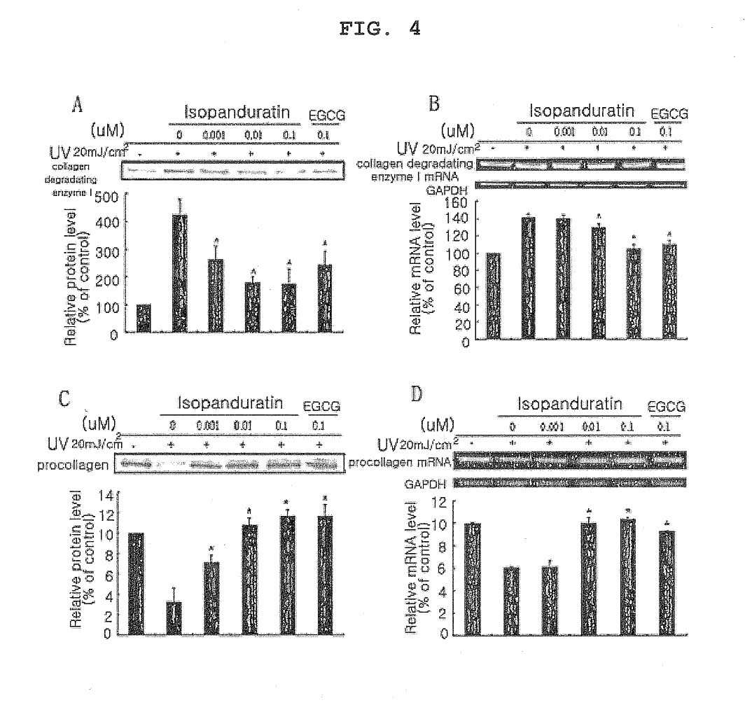

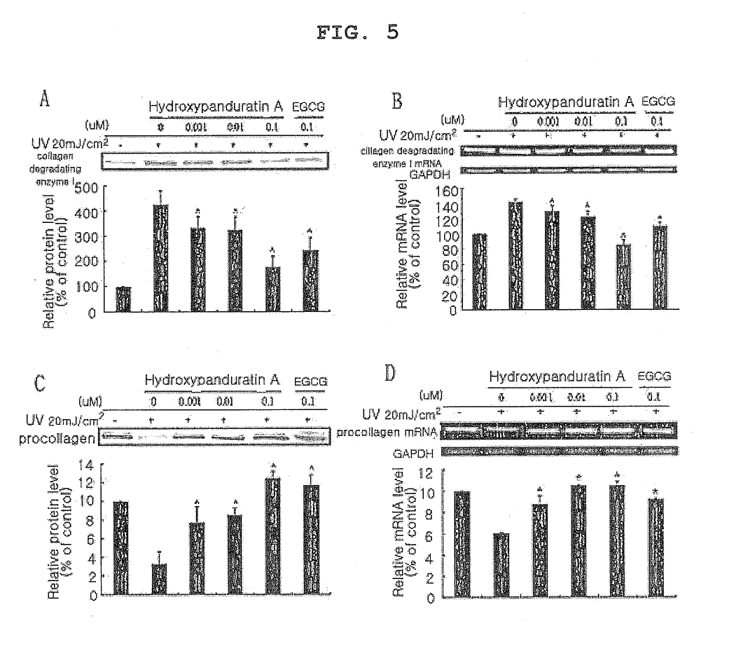

As seen from FIGS. 3-5, treatment with panduratin A (see FIG. 3), isopanduratin A (see FIG. 4) or 4-hydroxypanduratin A (see FIG. 5) resulted in the decrease of MMP-1 proteins and mRNAs in a concentration-dependent manner, whereas expression of procollagen proteins and mRNAs increased in a concentration-dependent manner. Particularly, they exhibited better activity than the control substance EGCG.

Thus, it was confirmed that the panduratin derivatives can be effectively used for anti-aging or wrinkle improvement because they inhibit the expression of collagenase and promotes procollagen synthesis.

### Test Example 3

### Collagen Degradation Inhibition Mechanism of Kaempferia pandurata Extract or Panduratin Derivatives

### 3-1. Effect on Activation of Mitogen-Activated Protein Kinases (MAPKs)

Human skin fibroblasts (CCD-986sk, ATCC, Manassas, Va., USA) were cultured in DMEM (Gibco, Grand Island, N.Y., USA). The fibroblasts were cultured on a 10 cm Petri dish (SPL, Seoul, Korea) to a concentration of 80% and further cultured for 24 hours in serum-free culture medium. Then, the cells were cultured for 24 hours in serum-free DMEM containing the panduratin A of Example 2. After replacing the culture medium with 5 mL of phosphate-buffered saline (PBS), the cells were exposed to UV (20 mJ/cm²). Cells that were not exposed to UV were used as negative control group, and cells treated with EGCG were used as positive control group.

The fibroblasts were lysed in RIPA buffer (Sigma-Aldrich Co., St. Louis, Mo., USA) and proteins were quantitated using a protein assay reagent (Bio-Rad Laboratories Inc., Hercules, Calif., USA). For Western blotting, the proteins were heated for 3 minutes and, after cooling, transferred to a nitrocellulose membrane (Amersham International, Little Chalfont, England) through electrophoresis in 10% SDS-PAGE. The membrane was saturated with 5% skim milk in TBST (10 mM Tris, pH 7.5, 100 mM NaCl, 0.1% Tween 20). After blotting for 2 hours using primary antibodies (diluted to 1:1000), followed by washing with TBST, blotting was performed for 2 hours using secondary antibodies (diluted to 1:2000). After washing with TBST for 3 times, the blotted antibodies were analyzed using an ECL detection system (Amersham International, Little Chalfont, England). The detection level was measured using the software RFLPscan version 2.1.

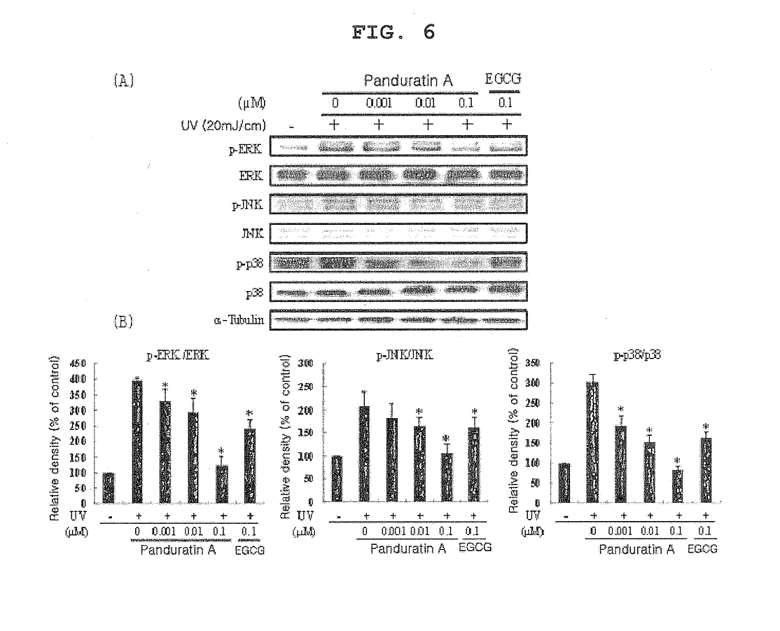

Through this procedure, the change of activity of extracellular-regulated protein kinase (ERK), Jun-N-terminal kinase (JNK) and p38 kinase, which belong to the MAPKs, was measured. The result is given in FIG. 6.

As seen from FIG. 6, ERK, JNK and p38 kinase were activated by phosphorylation when exposed to UV. But, the activation of ERK, JNK and p38 kinase was suppressed in a concentration-dependent manner when panduratin A was added. Considering that the activation of ERK, JNK and p38 kinase by phosphorylation induces the activation of activator protein-1 (AP-1) [Xu Y, Fisher G J., J. Dermatol. Sci. Suppl. 2005; 1: S1 S8], thereby promoting secretion of matrix metalloproteinases (MMPs) and degradation of collagen [Huang C, Schmid P C, Ma W Y, Schmid H H, Dong Z., J. Biol. Chem. 1997; 272: 4187 94], panduratin A may be effectively used for anti-aging or wrinkle improvement through inhibition of collagen degradation.

### 3-2. Effect on DNA Binding Activity of AP-1

Electrophoretic mobility shift assay (EMSA) was performed to measure the DNA binding activity of AP-1. Specifically, the fibroblasts, which were cultured in Test Example 3-1, to which panduratin A was added and which were exposed to UV, were washed with PBS and collected. After resuspending, them in 100 μL of lysis buffer (10 mM HEPES, 10 mM KCl, 0.1 mM EDTA, 1 mM DTT, 0.5 mM PMSF, pH 7.9) for 15 minutes, 30 μL of 5% NP-40 was added and mixed for 15 seconds. The cytosol component was removed by centrifuge and nuclear pellets were lysed using extraction buffer (20 mM HEPES, 0.4 M NaCl, 1 mM EDTA, 1 mM DTT, 1 mM PMSF, pH 7.9). Gel shift assay was carried out using quantitated nuclear proteins according to the manufacturer's instructions (Gel Shift Kit System; Panomics, Fremont, Calif., USA). Then, the detected proteins were analyzed with an ECL detection system (Amersham International, Little Chalfont, England) and the detection level was measured using the software RFLPscan version 2.1.

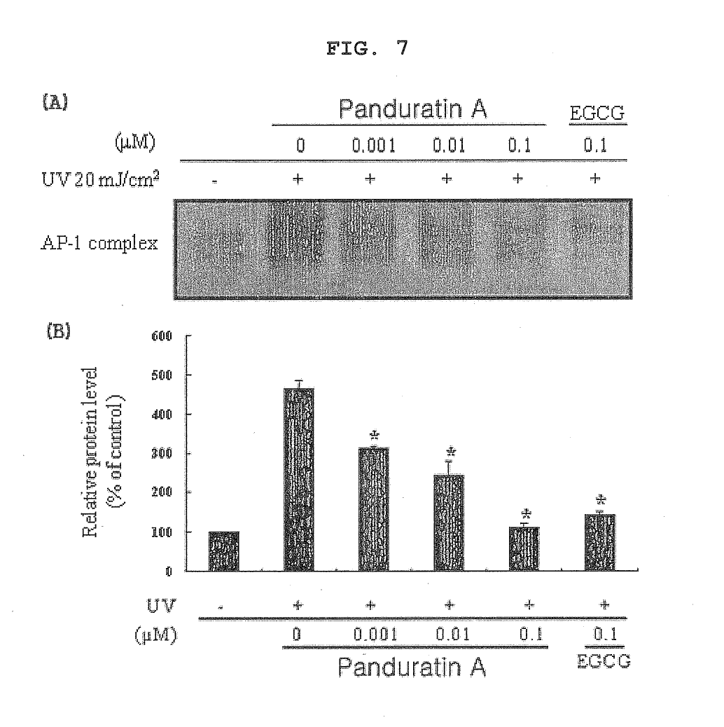

DNA binding activity of AP-1 was determined through this procedure. The result is given in FIG. 7.

As seen from FIG. 7, the addition of panduratin A resulted in inhibited binding of AP-1 to DNA in a concentration-dependent manner. Accordingly, considering that the activation of AP-1 may promote the secretion of MMPs and degradation of collagen, panduratin A may be effectively used for anti-aging or wrinkle improvement through inhibition of collagen degradation.

3-3. Effect on c-Jun and c-Fos Activity

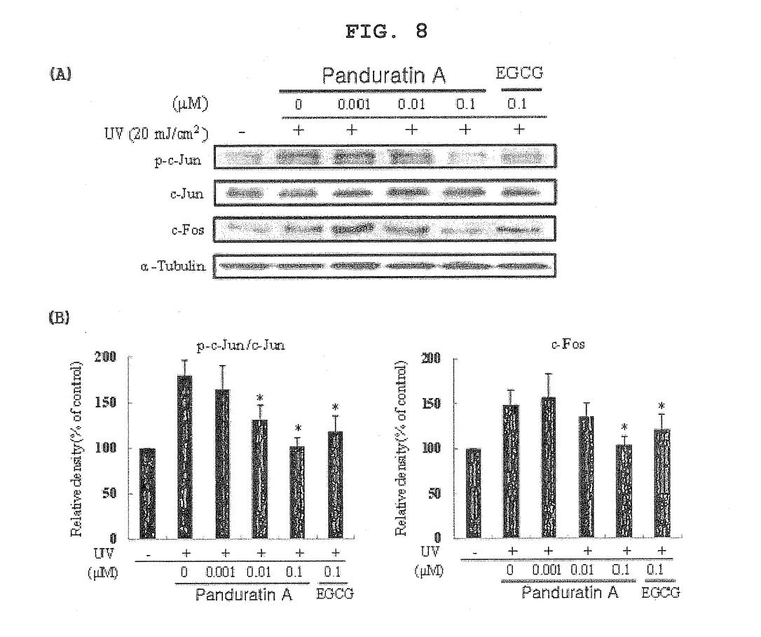

Western blotting was performed in the same manner as in Test Example 3-1 in order to investigate the effect of panduratin A on c-Jun and c-Fos activity. The result is given in FIG. 8.

As seen from FIG. 8, the addition of panduratin A resulted in inhibited c-Jun and c-Fos activity in a concentration-dependent manner. Considering the effect of c-Jun and c-Fos on transcriptional activity of AP-1 [Waskiewicz A J, Cooper J A., Curr. Opin. Cell Biol., 1995; 7: 798 805], it can be seen that the panduratin A may inhibit AP-1 activity, as in Test Example 3-2.

### Test Example 4

### Wrinkle Improvement Effect of Kaempferia pandurata Extract or Panduratin Derivatives

### 4-1. Application on Skin

Forty eight 6-week-old female hairless mice (Hos:
HR-1) were accustomed for a week and randomly divided into 6 groups, 8 per each. The hairless mice (Hos:HR-1) were exposed to UV for 8 weeks. UV irradiation was carried out 3 times a week, from 1 MED (1 MED=50 mJ/cm²) to 4 MED, until the end of the test. The 6 test groups were: non UV-treated group, UV-treated group, UV- and Kaempferia pandurata ethanol extract (0.1%, 0.5%)-treated group, and UV- and panduratin A (1 mM, 5 mM)-treated group. Each sample was dissolved in a mixture solvent of ethanol and polyethylene glycol (7:3, v/v) and 50 μL was applied on the back of the mice every day for 8 weeks. For the non UV-treated group and the UV-treated group, 50 μL of a mixture of ethanol and polyethylene glycol (7:3, v/v) was applied.

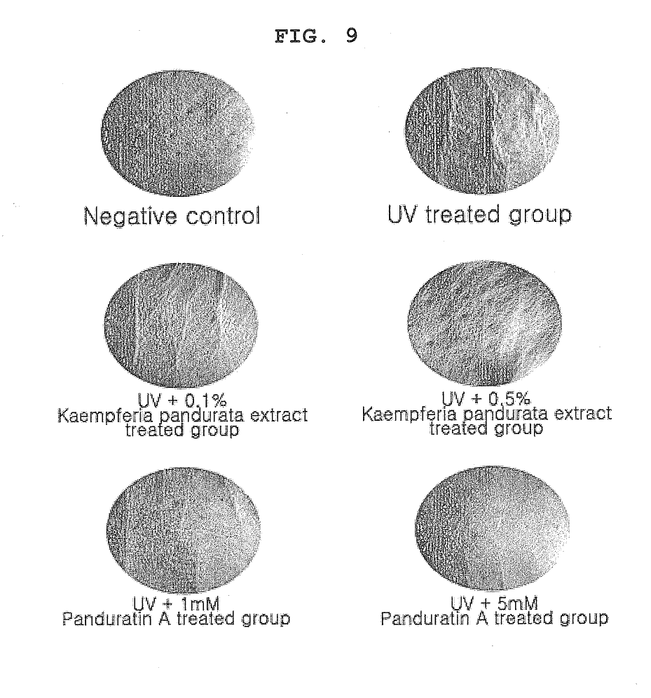

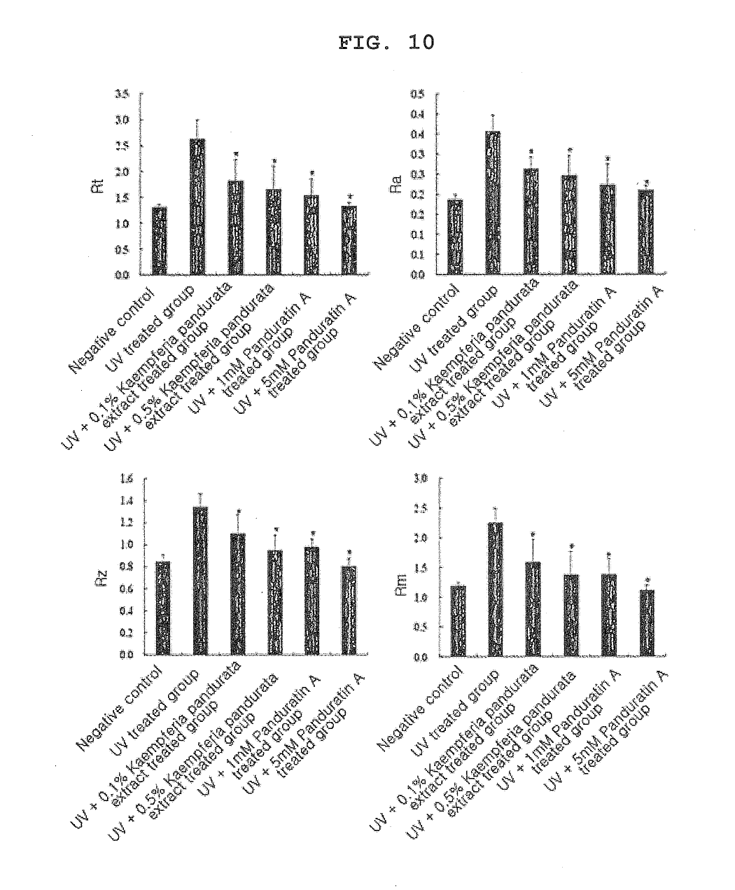

In order to investigate wrinkle prevention effect, skin replicas were taken using silicone polymer (SILFLO Impression Material, Flexico, England). The image files of the skin replicas were subjected to wrinkle evaluation using the computer image analysis software Skin Visiometer SV 600 (Courage+Khazaha Electronic, Kln, Germany). Rt, Rm, Rz and Ra values (Rt: the distance from the highest and lowest portions on the skin surface, Rm: the maximum Rt value of 5 measurements, Rz: the mean Rt value of 5 measurements, Ra: the arithmetic mean surface roughness) were determined. The result is given in FIGS. 9 and 10.

As seen from FIG. 9, the Kaempferia pandurata ethanol extract-treated group (0.1% and 0.5%) and the panduratin A-treated group (1 mM and 5 mM) exhibited remarkably decreased wrinkling as compared to the UV-treated group. Further, as seen from FIG. 10, both the Kaempferia pandurata ethanol extract-treated group and the panduratin A-treated group exhibited significantly decreased Rt, Rm, Rz and Ra values (p<0.05).

Accordingly, it can be seen that the application of the Kaempferia pandurata extract or the panduratin derivative on skin provides excellent wrinkle improvement effect.

### 4-2. Oral Administration

To the hairless mice exposed to UV in Test Example 4-1, the Kaempferia pandurata ethanol extract (200 mg/kg) or panduratin A (50 mg/kg) dissolved in 0.5% carboxymethyl cellulose solution containing 5% Tween 80 was orally administered every day for 8 weeks. For the control groups (non UV-treated group and UV-treated group), 0.5% carboxymethyl cellulose solution was administered.

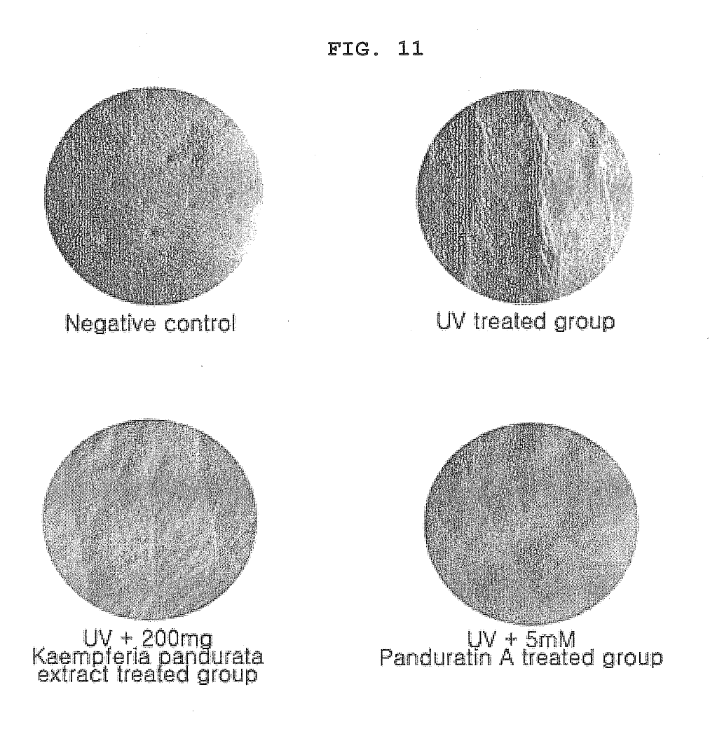

In order to investigate wrinkle prevention effect, skin replicas were taken using silicone polymer (SILFLO Impression Material, Flexico, England). The result is given in FIG. 11.

As seen from FIG. 11, the oral administration of the Kaempferia pandurata extract or the panduratin derivative exhibited remarkably decreased wrinkling.

Thus, it can be seen that the oral administration of the Kaempferia pandurata extract or the panduratin derivative provides excellent wrinkle improvement effect.

### Formulation Example 1

### Cosmetics

### 1-1 and 1-2. Nourishing Lotion (Milk Lotion)

Nourishing lotion was prepared according to a method commonly used in the related art using the panduratin derivative of Examples 2 through 4 or the Kaempferia pandurata extract of Example 1, with the compositions of the following Table 3.

TABLE 3
Nourishing lotion (milk lotion)

| | Formulation | Formulation |
|---|---|---|
| | Example 1-1 | Example 1-2 |
| | (wt %) | (wt %) |
| Panduratin derivative | 2.0 | — |
| Kaempferia pandurata | — | 2.0 |
| extract | | |
| Squalene | 5.0 | 5.0 |
| Beeswax | 4.0 | 4.0 |
| Polysorbate 60 | 1.5 | 1.5 |
| Sorbitan sesquioleate | 1.5 | 1.5 |
| Liquid paraffin | 0.5 | 0.5 |
| Caprylic/capric | 5.0 | 5.0 |
| triglyceride | | |
| Glycerine | 3.0 | 3.0 |
| Butylene glycol | 3.0 | 3.0 |
| Propylene glycol | 3.0 | 3.0 |
| Carboxyvinyl polymer | 0.1 | 0.1 |
| Triethanolamine | 0.2 | 0.2 |
| Antiseptic, pigment | adequate | adequate |
| and perfume | | |
| Purified water | to 100 | to 100 |

### 1-3 and 1-4. Softening Lotion (Skin Lotion)

Softening lotion was prepared according to a method commonly used in the related art using the panduratin derivative of Examples 2 through 4 or the Kaempferia pandurata extract of Example 1, with the compositions of the following Table 4.

TABLE 4
Softening lotion (skin lotion)

| | Formulation | Formulation |
|---|---|---|
| | Example 1-3 | Example 1-4 |
| | (wt %) | (wt %) |
| Panduratin derivative | 2.0 | — |
| Kaempferia pandurata | — | 2.0 |
| extract | | |
| Glycerine | 3.0 | 3.0 |
| Butylene glycol | 2.0 | 2.0 |
| Propylene glycol | 2.0 | 2.0 |
| Carboxyvinyl polymer | 0.1 | 0.1 |
| PEG 12 nonylphenyl | 0.2 | 0.2 |
| ether | | |
| Polysorbate 80 | 0.4 | 0.4 |
| Ethanol | 10.0 | 10.0 |
| Triethanolamine | 0.1 | 0.1 |
| Antiseptic, pigment | adequate | adequate |
| and perfume | | |
| Purified water | to 100 | to 100 |

### 1-5 and 1-6. Nourishing Cream

Nourishing cream was prepared according to a method commonly used in the related art using the panduratin derivative of Examples 2 through 4 or the Kaempferia pandurata extract of Example 1, with the compositions of the following Table 5.

TABLE 5
Nourishing cream

| | Formulation | Formulation |
|---|---|---|
| | Example 1-5 | Example 1-6 |
| | (wt %) | (wt %) |
| Panduratin derivative | 2.0 | — |
| Kaempferia pandurata | — | 2.0 |
| extract | | |
| Polysorbate 60 | 1.5 | 1.5 |
| Sorbitan sesquioleate | 0.5 | 0.5 |
| PEG 60 hydrogenated | 2.0 | 2.0 |
| castor oil | | |
| Liquid paraffin | 10 | 10 |
| Squalene | 5.0 | 5.0 |
| Caprylic/capric | 5.0 | 5.0 |
| triglyceride | | |
| Glycerine | 5.0 | 5.0 |
| Butylene glycol | 3.0 | 3.0 |
| Propylene glycol | 3.0 | 3.0 |
| Triethanolamine | 0.2 | 0.2 |
| Antiseptic | adequate | adequate |
| Pigment | adequate | adequate |
| Perfume | adequate | adequate |
| Purified water | to 100 | to 100 |

### 1-7 and 1-8. Massage Cream

Massage cream was prepared according to a method commonly used in the related art using the panduratin derivative of Examples 2 through 4 or the Kaempferia pandurata extract of Example 1, with the compositions of the following Table 6.

TABLE 6
Massage cream

| | Formulation | Formulation |
|---|---|---|
| | Example 1-7 | Example 1-8 |
| | (wt %) | (wt %) |
| Panduratin derivative | 1.0 | — |
| Kaempferia pandurata | — | 1.0 |
| extract | | |
| Beeswax | 10.0 | 10.0 |
| Polysorbate 60 | 1.5 | 1.5 |
| PEG 60 hydrogenated | 2.0 | 2.0 |
| castor oil | | |
| Sorbitan sesquioleate | 0.8 | 0.8 |
| Liquid paraffin | 40.0 | 40.0 |
| Squalene | 5.0 | 5.0 |
| Caprylic/capric | 4.0 | 4.0 |
| triglyceride | | |
| Glycerine | 5.0 | 5.0 |
| Butylene glycol | 3.0 | 3.0 |
| Propylene glycol | 3.0 | 3.0 |
| Triethanolamine | 0.2 | 0.2 |
| Antiseptic, pigment | adequate | adequate |
| and perfume | | |
| Purified water | to 100 | to 100 |

### 1-9 and 1-10. Pack

Pack was prepared according to a method commonly used in the related art using the panduratin derivative of Examples 2 through 4 or the Kaempferia pandurata extract of Example 1, with the compositions of the following Table 7.

TABLE 7
Pack

| | Formulation | Formulation |
|---|---|---|
| | Example 1-9 | Example 1-10 |
| | (wt %) | (wt %) |
| Panduratin derivative | 1.0 | — |
| Kaempferia pandurata | — | 1.0 |
| extract | | |
| Polyvinyl alcohol | 13.0 | 13.0 |
| Sodium carboxymethyl | 0.2 | 0.2 |
| cellulose | | |
| Glycerine | 5.0 | 5.0 |
| Allantoin | 0.1 | 0.1 |
| Ethanol | 6.0 | 6.0 |
| PEG 12 nonylphenyl | 0.3 | 0.3 |
| ether | | |
| Polysorbate 60 | 0.3 | 0.3 |
| Antiseptic, pigment | adequate | adequate |
| and perfume | | |
| Purified water | to 100 | to 100 |

### 1-11 and 1-12. Gel

Gel was prepared according to a method commonly used in the related art using the panduratin derivative of Examples 2 through 4 or the Kaempferia pandurata extract of Example 1, with the compositions of the following Table 8.

TABLE 8
Gel

| | Formulation | Formulation |
|---|---|---|
| | Example 1-11 | Example 1-12 |
| | (wt %) | (wt %) |
| Panduratin derivative | 0.5 | — |
| Kaempferia pandurata | — | 0.5 |
| extract | | |
| Ethylenediamine sodium | 0.05 | 0.05 |
| acetate | | |
| Glycerine | 5.0 | 5.0 |
| Carboxyvinyl polymer | 0.3 | 0.3 |
| Ethanol | 5.0 | 5.0 |
| PEG 60 hydrogenated | 0.5 | 0.5 |
| castor oil | | |
| Triethanolamine | 0.3 | 0.3 |
| Antiseptic, pigment | adequate | adequate |
| and perfume | | |
| Purified water | to 100 | to 100 |

### Formulation Example 2

### Foods

### 2-1. Health Food

1,000 mg of the panduratin derivative of Examples 2 through 4 or the Kaempferia pandurata extract of Example 1 may be mixed with 70 μg of vitamin A acetate, 1.0 mg of vitamin E, 0.13 mg of vitamin B₁, 0.15 mg of vitamin B₂, 0.5 mg of vitamin B₆, 0.2 μg of vitamin B₁₂, 10 mg of vitamin C, 10 μg of biotin, 1.7 mg of nicotinamide, 50 μg of folic acid, 0.5 mg of calcium pantothenate, 1.75 mg of ferrous sulfate, 0.82 mg of zinc oxide, 25.3 mg of magnesium carbonate, 15 mg of monobasic potassium phosphate, 55 mg of dibasic calcium phosphate, 90 mg of potassium citrate, 100 mg of calcium carbonate and 24.8 mg of magnesium chloride. The mixing proportion may be changed differently. The mixture may be prepared into granules according to a method commonly used in the related art and may be used for the preparation of a health food composition according to a method commonly used in the related art.

### 2-2. Health Drink

1,000 mg of the panduratin derivative of Examples 2 through 4 or the Kaempferia pandurata extract of Example 1 may be mixed with 1,000 mg of citric acid, 100 g of oligosaccharide, 2 g of plum extract and 1 g of taurine according to a method commonly used in the related art. Purified water may be added to a total volume of 900 mL. After heating at 85° C. for about 1 hour while stirring, the resultant solution may be filtered and collected in a sterilized 2 L container. After sealing and sterilization, it may be kept cold to prepare a health drink composition.

### 2-3. Chewing Gum

0.1 wt % of the panduratin derivative of Examples 2 through 4 or the Kaempferia pandurata extract of Example may be mixed with 20 wt % of gum base, 76.9 wt % of sugar, 1 wt % of perfume and 2 wt % of water according to a method commonly used in the related art to prepare chewing gum.

2-4. Candy

0.1 wt % of the panduratin derivative of Examples 2 through 4 or the Kaempferia pandurata extract of Example 1 may be mixed with 60 wt % of sugar, 39.8 wt % of starch syrup and 0.1 wt % of perfume according to a method commonly used in the related art to prepare candy.

### 2-5. Biscuit

1 wt % of the panduratin derivative of Examples 2 through 4 or the Kaempferia pandurata extract of Example 1 may be mixed with 25.59 wt % of hard wheat flour, 22.22 wt % of medium wheat flour, 4.80 wt % of refined sugar, 0.73 wt % of table salt, 0.78 wt % of glucose, 11.78 wt % of palm shortening, 1.54 wt % of ammonium, 0.17 wt % of baking soda, 0.16 wt % of sodium bisulfite, 1.45 wt % of rice flour, 0.0001 wt % of vitamin B₁, 0.0001 wt % of vitamin B₂, 0.04 wt % of milk flavor, 20.6998 wt % of water, 1.16 wt % of whole milk powder, 0.29 wt % of milk replacer, 0.03 wt % of monobasic calcium phosphate, 0.29 wt % of scattering salt and 7.27 wt % of spray-milk according to a method commonly used in the related art to prepare biscuit.

### Formulation Example 3

### Drugs

### 3-1. Powder

50 mg of the panduratin derivative of Examples 2 through 4 or the Kaempferia pandurata extract of Example 1 may be mixed with 2 g of crystalline cellulose and put in an airtight pouch according to a method commonly used in the related art to prepare powder.

### 3-2. Tablet

50 mg of the panduratin derivative of Examples 2 through 4 or the Kaempferia pandurata extract of Example 1 may be mixed with 400 mg of crystalline cellulose and 5 mg of magnesium stearate and prepared into tablet according to a method commonly used in the related art.

### 3-3. Capsule

30 mg of the panduratin derivative of Examples 2 through 4 or the Kaempferia pandurata extract of Example 1 may be mixed with 100 mg of whey protein, 400 mg of crystalline cellulose and 6 mg of magnesium stearate and filled in a gelatin capsule according to a method commonly used in the related art to prepare capsule.

### 3-4. Injection

According to a method commonly used in the related art, active ingredients may be dissolved in distilled water for injection and pH may be adjusted to about 7.5. Then, 100 mg of the panduratin derivative of Examples 2 through 4 or the Kaempferia pandurata extract of Example 1, distilled water for injection and pH adjuster may be mixed and filled in a 2 mL ampule and sterilized to prepare injection.

### INDUSTRIAL APPLICABILITY

The said the panduratin derivative or the Kaempferia pandurata extract comprising the same induces cell proliferation, inhibits degradation of collagen, and promotes synthesis of collagen, therefore, it shows excellent activity in prevention of aging, particularly preventing, improving or treating wrinkle and it can be used as an effective ingredient in a cosmetic, food or pharmaceutical composition.

1. A cosmetic composition for wrinkle improvement and/or anti-aging comprising a panduratin derivative selected from the group consisting of the compounds represented by the following Chemical Formulas 1 to 3 or a Kaempferia pandurata extract comprising the panduratin derivative.

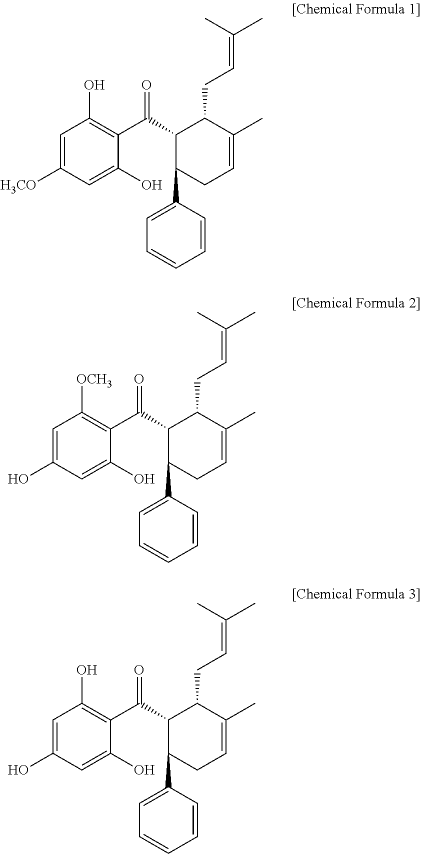

2. A food composition for wrinkle improvement and/or anti-aging comprising a panduratin derivative selected from the group consisting of the compounds represented by the following Chemical Formulas 1 to 3 or a Kaempferia pandurata extract comprising the panduratin derivative.

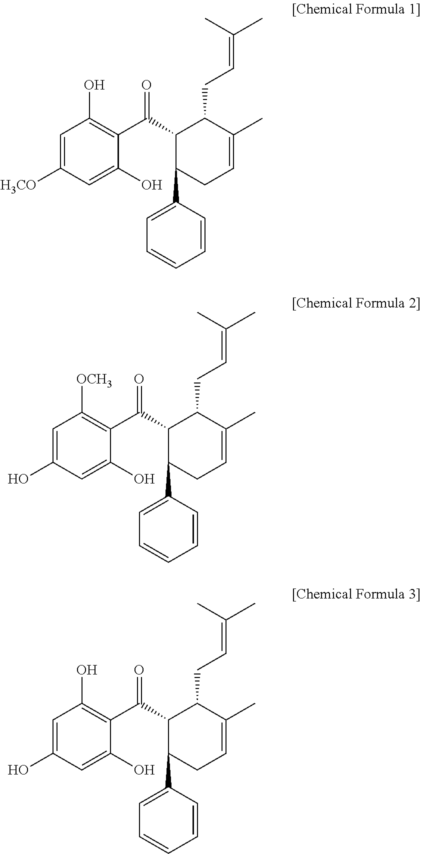

3. A pharmaceutical composition for preventing and treating wrinkle and/or preventing aging comprising a panduratin derivative selected from the group consisting of the compounds represented by the following Chemical Formulas 1 to 3 or a Kaempferia pandurata extract comprising the panduratin derivative.

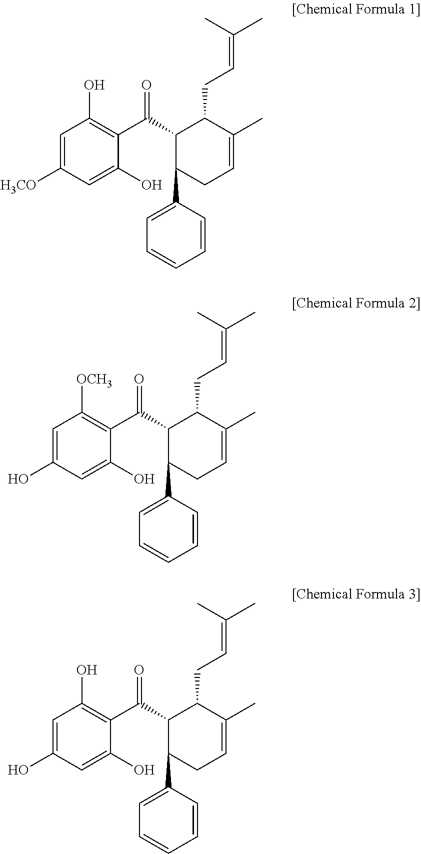

4. The composition of anyone selected from the group consisting of claims 1 to 3, wherein the Kaempferia pandurata extract is extracted with a solvent which is selected from the group consisting of water, C1-C6 organic solvent and subcritical or supercritical fluid.

5. The composition of claim 4, wherein the C1-C6 organic solvent is one which is selected from the group consisting of C1-C6 alcohol, acetone, ether, benzene, chloroform, ethyl acetate, methylene chloride, hexane, cyclohexane, and petroleum ether.

6. Use of a panduratin derivative selected from the group consisting of the compounds represented by the following Chemical Formulas 1 to 3 or a Kaempferia pandurata extract comprising the panduratin derivative for preparing a cosmetic composition.

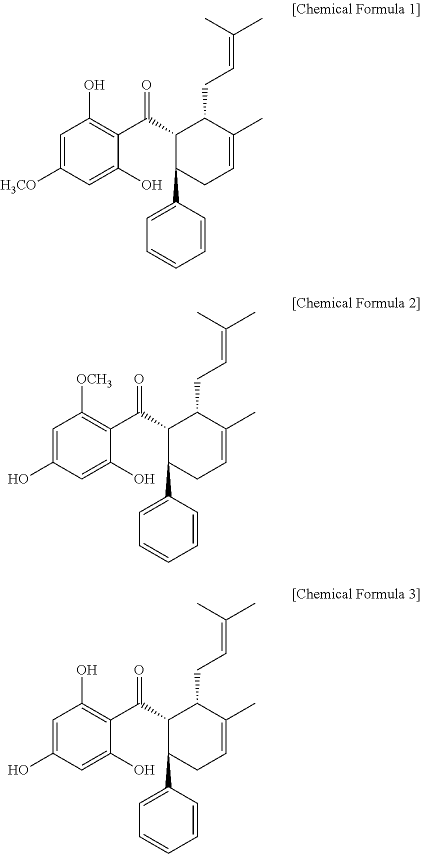

7. Use of a panduratin derivative selected from the group consisting of the compounds represented by the following Chemical Formulas 1 to 3 or a Kaempferia pandurata extract comprising the panduratin derivative for preparing a food composition.

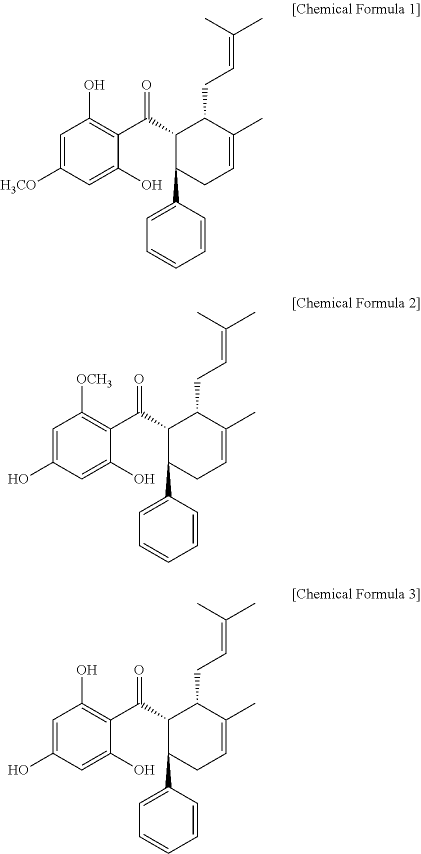

8. Use of a panduratin derivative selected from the group consisting of the compounds represented by the following Chemical Formulas 1 to. 3 or a Kaempferia pandurata extract comprising the panduratin derivative for preparing a pharmaceutical composition.

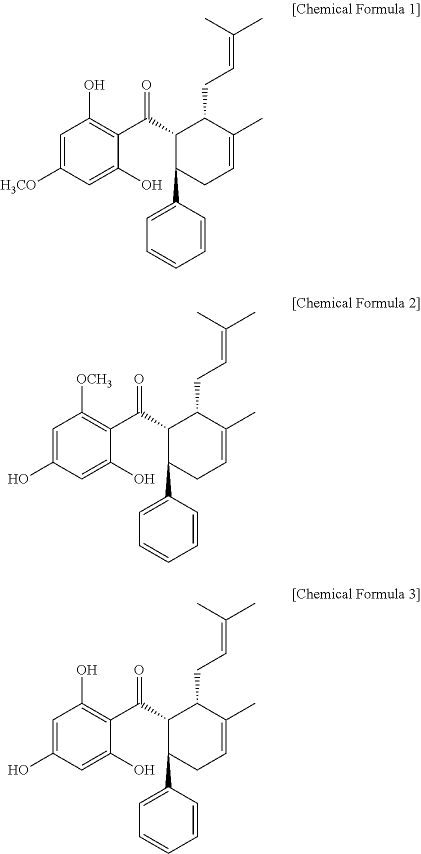

9. A method for preventing, improving or treating wrinkle of a panduratin derivative selected from the group consisting of the compounds represented by the following Chemical Formulas 1 to 3 or a Kaempferia pandurata extract comprising the panduratin derivative.

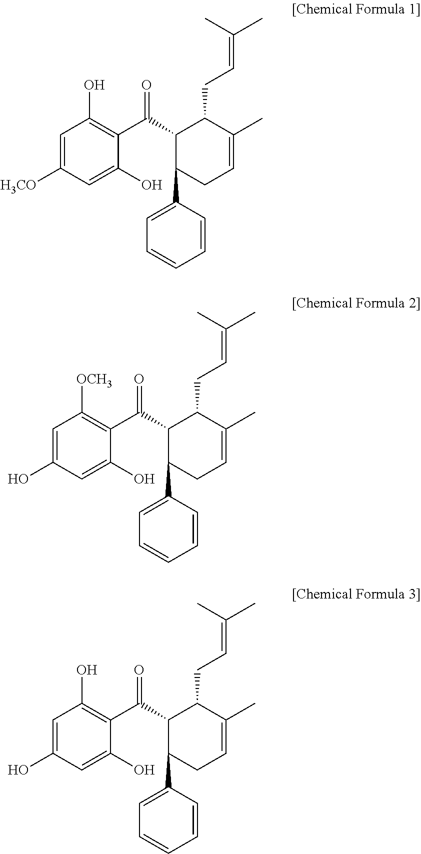

10. A method for preventing aging of a panduratin derivative selected from the group consisting of the compounds represented by the following Chemical Formulas 1 to 3 or a Kaempferia pandurata extract comprising the panduratin derivative.

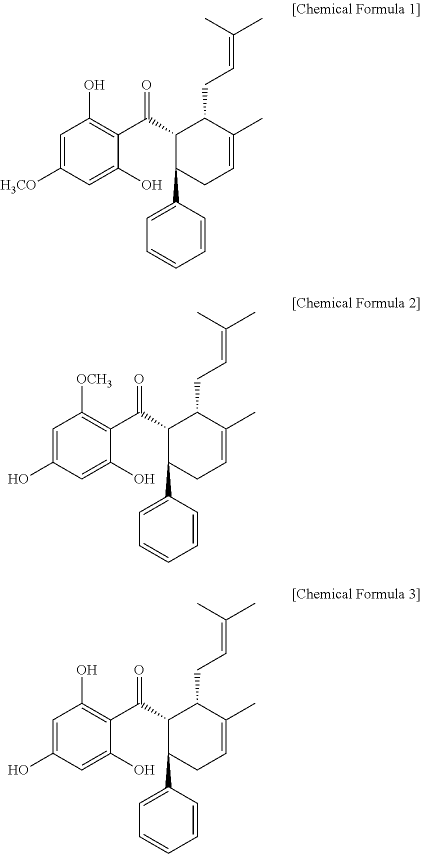

11. A method for promoting collagen synthesis of a panduratin derivative selected from the group consisting of the compounds represented by the following Chemical Formulas 1 to 3 or a Kaempferia pandurata extract comprising the panduratin derivative.

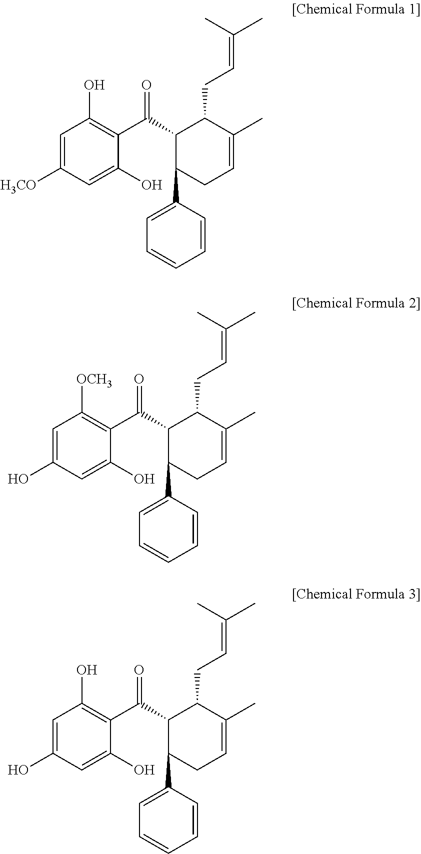

12. A method for inhibiting collagen degradation of a panduratin derivative selected from the group consisting of the compounds represented by the following Chemical Formulas 1 to 3 or a Kaempferia pandurata extract comprising the panduratin derivative. [chemical structure]
